(19) United States
(12) Patent Application Publication
(10) Pub. No.: US 2023/0397830 A1
(43) Pub. Date: Dec. 14, 2023

(54) HEARING SYSTEM AND METHOD FOR MEASURING THE PULSE OF A PERSON BY USING A HEARING SYSTEM

(71) Applicant: Sivantos Pte. Ltd., Singapore (SG)

(72) Inventor: Umut Gökay, Erlangen (DE)

(21) Appl. No.: 18/334,506

(22) Filed: Jun. 14, 2023

(30) Foreign Application Priority Data
Jun. 14, 2022 (DE) 10 2022 206 012.1

(51) Int. Cl. A61B 5/0255; H04R 1/10; H04R 19/02; A61B 5/00

(52) U.S. Cl. CPC A61B 5/0255; H04R 1/1091; H04R 19/02; A61B 5/6815

(57) ABSTRACT

A method for measuring the pulse of a person uses a hearing system having at least one hearing instrument with an electroacoustic first input transducer. A first structure-borne sound signal at an ear of the person is picked up by the first input transducer, and a first input signal is generated as a result. A pulse rate is determined based on an amplitude profile of the first input signal. A corresponding hearing system is configured to measure a pulse according to the method.

### CROSS-REFERENCE TO RELATED APPLICATION

This application claims the priority, under 35 U.S.C. § 119, of German Patent Application DE 10 2022 206 012.1, filed Jun. 14, 2022; the prior application is herewith incorporated by reference in its entirety.

### FIELD AND BACKGROUND OF THE INVENTION

The invention relates to a method for measuring the pulse of a person by using a hearing system. The invention also relates to a hearing system which is set up for carrying out such a method.

A hearing instrument should be generally understood as meaning a device which is set up to generate an output sound, which is delivered to the hearing of a wearer, from an electrical audio signal (which may also take the form of an internal processing signal of the device) by an electroacoustic output transducer (for example a loudspeaker). The hearing instrument may in that case be configured for example as headphones, in particular as earplug-like headphones. The hearing instrument may however also take the form of a hearing device “in the narrower sense,” which is intended and set up to correct a hearing impairment of the wearer, in that an ambient sound is converted by at least one electroacoustic input transducer (for example a microphone) into an input signal, which is processed according to the audiological requirements of the wearer and thereby amplified, in particular frequency-band specifically. An audio signal resulting from the processing is then converted by an electroacoustic output transducer of the hearing instrument into an output sound.

Hearing systems, that is to say systems with at least one hearing instrument, are increasingly being provided with the function of measuring the heart rate of a wearer of the hearing system. That is on the one hand the case for headphones with a fitness function, which the wearer wears during a sporting activity. The headphones may in that case also monitor further physiological parameters, or the hearing system may additionally also include a smartwatch or the like, through the use of which further such parameters can be monitored. Moreover, the wearer can for example listen to music during the sporting activity as a diversion. On the other hand, hearing devices in the narrower sense may also have such functions. In that case, account is thereby taken in particular of the fact that hearing devices are not uncommonly worn by elderly persons, for whom health monitoring is desired or even indicated.

Measuring of the heart rate may be performed by a hearing instrument for example by using photoplethysmography (PPG). A PPG sensor thereby emits light of different wavelengths into the tissue by using one or more LEDs, and subsequently detects substantially the light transmitted and/or reflected by the tissue. That light, which partly propagates through blood vessels under the surface of the skin, is in that case subject to the periodic variations of the pulse beat, so that the heart rate can be determined on the basis of those variations.

One disadvantage however is that, with permanent use, the pulse measurement by using PPG described herein results in a high energy consumption for the LEDs being used. However, especially in the case of hearing instruments, the battery power available for operation is often very limited.

### SUMMARY OF THE INVENTION

It is accordingly an object of the invention to provide a hearing system and a method for measuring the pulse of a person by using a hearing system with at least one hearing instrument, which overcome the hereinafore-mentioned disadvantages of the heretofore-known systems and methods of this general type and with which measuring the pulse of a wearer of the hearing system can be carried out as energy-efficiently and reliably as possible.

With the foregoing and other objects in view there is provided, in accordance with the invention, a method for measuring the pulse of a person by using a hearing system, the hearing system including at least a first hearing instrument with an electroacoustic first input transducer, in which a first structure-borne sound signal is picked up by the first input transducer at an ear of the person, and a first input signal is generated as a result. According to the method, it is provided in this case that a pulse rate is determined on the basis of an amplitude profile of the first input signal.

Configurations that are advantageous and in some cases inventive in themselves are presented in the subclaims and in the description that follows.

Preferably, it may in this case be provided that, at least for a time, in particular in a special mode still to be defined, an auxiliary signal is generated by a first sensor of the hearing system and/or a second input transducer of the hearing system, a first item of information about a pulse beat of the person is obtained on the basis of the auxiliary signal, and then the pulse rate is determined on the basis of the first item of information and on the basis of the first input signal.

In particular, the pulse rate may also be determined on the basis of the first item of information, the first item of information including at least two extremes and/or at least two rising or falling flanks of an envelope of the first input signal and/or an absolute value function of the first input signal. Consequently, the first item of information can therefore be obtained on the basis of the auxiliary signal that is generated by the first sensor and/or a second input transducer, or else from the profile of the first input signal itself.

With the objects of the invention in view, there is concomitantly provided a hearing system, which comprises at least one hearing instrument with an electroacoustic first input transducer, a control unit, and also preferably furthermore a first sensor and/or a second input transducer, the hearing system being set up to carry out the previously described method for measuring the pulse of a person when at least the first hearing instrument is worn, in particular as-intended, at an ear of the person. In particular, the first input transducer is set up in this case to pick up a first structure-borne sound signal at an ear of the person, and to generate a first input signal as a result, and the control unit being set up to determine a pulse rate on the basis of an amplitude profile of the first input signal. In particular, the first sensor of the hearing system or the second input transducer of the hearing system may in this case be set up to generate an auxiliary signal, which provides information on the pulse beat of the person, so that, at least for a time, a first item of information about the pulse beat of the person can be obtained on the basis of the auxiliary signal, and then the control unit also determines the pulse rate on the basis of the first item of information (and on the basis of the first input signal).

The hearing system according to the invention shares the benefits of the method according to the invention, and is set up in particular for carrying out the method by a corresponding construction and configuration. The advantages indicated for the method and for its developments can be transferred, mutatis mutandis, to the hearing system.

A hearing instrument generally includes any device which is intended and set up to generate from an electrical output signal by using an electroacoustic output transducer a corresponding output sound, and to deliver this to the hearing of a user. A loudspeaker may in particular be used in this case as such an output transducer, but it is also possible for example for thermoacoustic transducers to be used. A hearing instrument may in this case be configured on the one hand just for generating the output sound on the basis of audio data, that is to say for example in the form of wireless headphones, in particular in the form of earplugs. In this case, an output sound is generated on the basis of audio data, which may for example take the form of music, and which have been stored in advance, or else be transmitted to the hearing instrument by way of a corresponding antenna (streaming).

A hearing instrument may however also take the form of a hearing device (“in the narrower sense”), which is set up to correct or at least partially compensate for a hearing impediment of a user, in that for example an ambient sound is converted by using at least one electroacoustic input transducer, such as for example a microphone (or multiple microphones), into a corresponding electrical input signal, which is processed in the hearing device according to the audiological requirements of the wearer and thereby amplified, in particular in terms of the frequency band, so that the processed input signal is delivered by way of the electroacoustic output transducer to the hearing of the user as output sound.

An electroacoustic input transducer includes in this case in particular such a transducer that is set up to generate a corresponding electrical signal from the ambient sound. In particular, when generating the first or second input signal by the respective input transducer, a preprocessing may also take place, for example in the form of a linear pre-amplification and/or an A/D conversion. The correspondingly generated input signal in this case takes the form in particular of an electrical signal, the current and voltage fluctuations of which substantially represent the sound pressure fluctuations of the air.

The first structure-borne sound signal recorded by the first input transducer at the ear of the person preferably contains a sound component which corresponds to a pulse beat or can be assigned to such a pulse beat. The first hearing instrument is in this case worn by the person in or at an ear, so that the first structure-borne sound signal is recorded by the first input transducer of the first hearing instrument in or at the ear. Correspondingly, the first structure-borne sound signal contains sound components of a pulse beat in the region of the ear concerned, preferably in the auditory canal. For this, preferably the first input transducer is disposed in or on the first hearing instrument in such a way that, when the first hearing instrument is worn as intended, it is directed into the auditory canal, which is at least partially closed by the first hearing instrument. The first input transducer in this case preferably also records the first structure-borne sound signal of the occluded auditory canal, i.e. the pulse beat of the blood vessels surrounding the auditory canal generates in the (at least partially) closed auditory canal a pulsating noise which cannot (completely) escape as a result of the occlusion, and consequently is included in the first structure-borne sound signal as a corresponding sound component and, as a result, is also included in the first input signal.

Included as the first sensor of the hearing system is in particular a PPG sensor, but also an acceleration sensor disposed on a carotid artery, or an ECG sensor with corresponding electrodes, which are disposed at a suitable point on the body of the person during the operation of the hearing system. In the first-mentioned case of the PPG sensor, it is preferably disposed in the first hearing instrument. The hearing system may however also include one or more further devices, such as for example a smartwatch or the like, which can be connected data-technically to the first hearing instrument. The first sensor may then be disposed in such a further device.

The first sensor is in this case preferably set up to generate as the auxiliary signal a sensor signal on the basis of which the first item of information about the pulse beat of the person can be obtained. In particular, the sensor signal (that is to say the auxiliary signal) can make an independent pulse measurement possible, or at least allow an inference of a pulse rate and/or a phase of a pulse beat.

In particular, however, a second input signal of an electroacoustic second input transducer of the hearing system may also be generated as the auxiliary signal. In this case, the second input transducer is in particular disposed in a second hearing instrument of a binaural hearing system. The generation of the second input signal as an auxiliary signal then takes place in a comparable way to the first input signal, a second structure-borne sound signal being recorded.

On the basis of the first item of information, which preferably includes a reference pulse rate and/or a phase of a pulse beat, a measurement of the pulse rate can then be carried out directly in the first input signal. This may take place for example by a correlation measurement or for instance also by a so-called “minimum tracking” of the signal amplitude, the first item of information being additionally used as a reference.

This is so because, if a signal-to-noise ratio (SNR) of the sound component of the pulse beat in the first input signal is disadvantageous, it is additionally possible to revert to the reference of the first item of information for determining the first pulse rate. In this case it is possible for the auxiliary signal in particular only to be used when it is actually required, because for example there is unfavorable SNR for a pulse measurement in the first input signal.

In particular, it is therefore also possible in this case for an independent pulse measurement to be carried out on the basis of an auxiliary signal from a PPG measurement or the like. In the further course of the procedure, the pulse rate determined in this way as a first item of information is then only used for determining the pulse rate on the basis of the signal components of the first input signal as a reference (for example for a correlation measurement).

Preferably, in a normal mode, the pulse rate is determined on the basis of the first input signal, in particular on the basis of the amplitude profile and particularly preferably without generating and/or without using current values of the auxiliary signal, whereas, in the event of an error when determining the pulse rate in the normal mode, a change is made to a special mode, and whereas, in the special mode, the first item of information about a pulse beat of the person is obtained on the basis of the auxiliary signal or on the basis of a correlation measurement, and the pulse rate is determined on the basis of the first item of information (and possibly on the basis of the first input signal). This means in particular that, in the normal operation of the hearing system, the measurement signal relevant for the pulse measurement is the first input signal, which carries the current information about the pulse beat. The measurement of the pulse rate is then only carried out on the current first input signal, but possibly on the basis of additional items of information that have been obtained at a previous point in time on the basis of the auxiliary signal. For this reason, detection of the auxiliary signal is not required in the normal mode.

Only in the event of an error, that is to say in particular if the pulse rate cannot be determined from the first input signal in the normal mode (for instance because the SNR is too poor, or because a reference has been lost because of a change in the physical activity of the person), the first item of information is obtained from the auxiliary signal, and consequently a change is made to the special mode.

Preferably, then, after a successful determination of the pulse rate in the special mode, a change is made back to the normal mode, that is to say in particular obtainment of the first item of information and preferably also detection of the auxiliary signal are suspended. If the auxiliary signal takes the form of a second input signal of the hearing system that is continuously generated while the hearing system is operating (and in particular is also put to some other use during operation), when a return is made to normal mode in particular use of the current values of the auxiliary signal for pulse measurement is suspended. This allows particularly energy-efficient measurement of the pulse rate, since the increased precision of the auxiliary signal is only used (or the first item of information is only obtained) when it is required, to be specific when a pulse measurement in the normal mode first fails, and consequently a change to the special mode is made. The higher energy consumption in the special mode as a result of the first sensor is then only maintained until the pulse rate can again be detected with the aid of the reference of the first item of information.vv

Preferably, after the change from the special mode to the normal mode, the pulse rate is in this case determined on the basis of the first input signal and the first item of information obtained in the previous special mode. This includes in particular that for example, after a return to the normal mode, a pulse rate determined in the special mode on the basis of an auxiliary signal from a PPG measurement is used as a reference for measuring the pulse rate just on the basis of the current signal contributions of the first input signal, and the PPG measurement is in this case suspended in the normal mode.

Expediently, the pulse rate is determined (at least in the normal mode) on the basis of a correlation measurement of the first input signal, a parameter of the correlation measurement being determined in particular on the basis of the first item of information.

In this case, in particular, the correlation of the first input signal may be determined by a test function, which is assigned a predetermined frequency or which has a predetermined frequency. In this case, the test function preferably models a specific signal profile, in particular of the first input signal, for a pulse beat of the predetermined frequency. For this, the test function should preferably be selected on the basis of the first item of information from a plurality of possible test functions of different frequencies and possibly also different signal profiles. Then, in particular the frequency of the test function may be predetermined on the basis of the first item of information as a parameter of the correlation measurement. In particular, for the plurality of different possible test functions with different frequencies, in this case a correlation of the first input signal with a test function may be respectively determined from the plurality, the first test function being determined from the plurality on the basis of a maximum of the correlations. The first test function is consequently in particular the test function from the plurality of possible test functions that has the highest correlation with the first input signal.

Preferably, in this case in the special mode the first test function is determined from the plurality of test functions and/or the frequency of the first test function is determined on the basis of the maximum of the correlations as the first item of information. In other words, this means in particular that, in the special mode, the first item of information is given by the first test function itself or by the identification of the first test function on the basis of its frequency.

Favorably, the first item of information is obtained when the value of the correlation measurement falls below a predetermined limit value. In particular, in this case the value of the correlation measurement may also be used as a criterion for a change from the normal mode to the special mode. If, for example, the correlation measurement is formed as a crosscorrelation of the first input signal with the predetermined test function, the phase of the respectively current pulse cycle can be determined on the basis of the maximum by way of the crosscorrelation argument. If, however, this maximum falls below the predetermined limit value, it can be assumed that the correlation between the predetermined test function of a fixed frequency and the first input signal is no longer sufficiently high, and correspondingly a different test function should be selected.

In an advantageous configuration, in the normal mode, a correlation measurement with the first input signal is respectively carried out for a group of test functions of which the frequencies form a corridor around the frequency of the first test function, with an updating of the frequency of the first test function taking place on the basis of a maximum of the correlation measurements. This includes in particular that, in the normal mode, the first test function (or its frequency) is therefore determined at a first point in time, and then a “corridor” of further test functions is chosen around the first test function (that is to say the group of test functions with frequencies in a corridor, that is to say in particular an interval about the frequency of the first test function), the corridor forming in particular a genuine subset of all available test functions, and the correlation with the first input signal being respectively determined for these test functions. As a result, when there are slow changes in the pulse rate, a deviation can be easily established, and a new first test function from the corridor can be defined. The corridor can then be correspondingly shifted.

Expediently, the first input transducer picks up the first structure-borne sound signal in the auditory canal at the ear of the person. In this case, the first input transducer may be intended and set up in particular for active occlusion suppression, which is used in the hearing instrument to compensate for excessively loud structure-borne sound in the auditory canal.

In a development, an electroacoustic second input transducer of a second hearing instrument of the hearing system picks up a second structure-borne sound signal at the other ear of the person, from which the auxiliary signal is generated as a second input signal. Preferably, in this case a correlation measurement of the first and second input signals is carried out, and the pulse rate is determined on the basis of the value of the correlation resulting from this. This means in particular that in the normal mode the pulse rate is determined from the current values of the first input signal, and only in the special mode are the current values of the second input signal used. Using a second input signal is appropriate in particular for a binaural hearing system, since such a second input signal can be obtained from the contramedial hearing instrument, where it is used for example for active occlusion suppression. This is so because it can be assumed that a loss of the pulse rate in a measurement at one ear (for instance because of an inadequate SNR there) proceeds independently of the measurement at the other ear, so that the pulse rate measured at the other rear should still be stable and meaningful, and therefore can serve as a reference for the first-mentioned, unstable measurement.

In a further advantageous configuration, a PPG sensor is used as the first sensor, a pulse measurement being carried out on the basis of the auxiliary signal generated by the PPG sensor, with a reference pulse rate of the person being determined as a first item of information, and then this first item of information being used as a reference for the subsequent determination of the pulse rate on the basis of the first input signal, in particular only on the basis of the first input signal. Preferably, in this case the reference pulse rate thus determined is used as a reference for the correlation measurement, the correlation measurement of the first input signal taking place in particular without current signal components of the auxiliary signal after the determination of the reference pulse rate as the first item of information.

In an advantageous configuration, an acceleration sensor which is disposed on a carotid artery of the person, and/or an electrocardiogram sensor (in this case an electrocardiogram of the person is created as the auxiliary signal), is used as the first sensor, with a reference pulse rate of the person being determined as a first item of information on the basis of a movement of the carotid artery recorded by the acceleration sensor or from the electrocardiogram, and then this first item of information is used as a reference for a subsequent determination of the pulse rate on the basis of the first input signal, in particular only on the basis of the first input signal. The sensors likewise provide information about the pulse beat, and can moreover likewise be used in a hearing system, so that they do not have to be additionally disposed in a dedicated manner.

In particular, the method may also be used for determining a further or other cardiovascular variable, that is to say for example a blood pressure or a difference between systolic and diastolic blood pressure (so-called blood pressure amplitude) of a wearer of the hearing instrument. Especially the last-mentioned value as a cardiovascular variable can provide information on possible risks of cardiovascular diseases. In particular, a contrast intensity may be used for this purpose.

Other features which are considered as characteristic for the invention are set forth in the appended claims.

Although the invention is illustrated and described herein as embodied in a hearing system and a method for measuring the pulse of a person by using a hearing system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, longitudinal-sectional view of a hearing device having a block circuit diagram set up for measuring the pulse of its wearer; and

FIG. 2 is a block diagram illustrating the sequence of a pulse measurement by using the hearing device shown in FIG. 1.

### DETAILED DESCRIPTION OF THE INVENTION

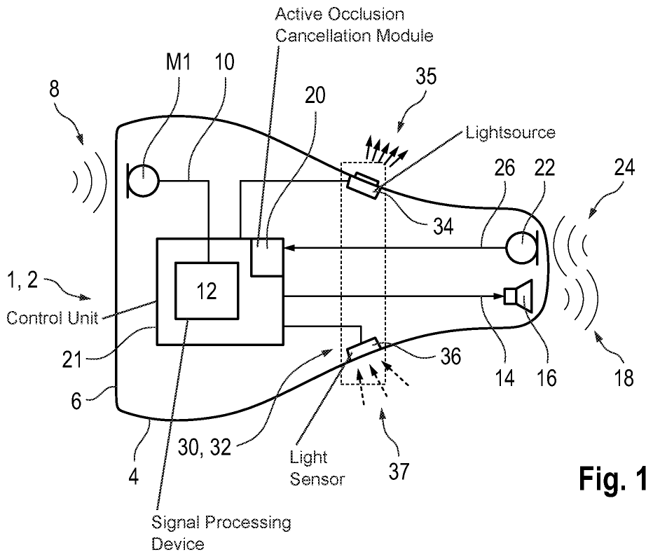

Referring now in detail to the figures of the drawings, in which parts and variables corresponding to one another are respectively provided with the same designations, and first, particularly, to FIG. 1 thereof, there is seen a diagrammatically illustrated hearing instrument 1, which in the present case is configured as a hearing device 2 (in the narrower sense), and includes a block circuit diagram. The hearing device 2 may on the one hand be of a monaural configuration (that is to say it may take the form of a stand-alone hearing device), or it may be part of a binaural hearing system with a further hearing device (not shown). Similarly, the hearing device 2 may be part of a hearing system that is not shown any more specifically, which also includes at least one auxiliary device (not shown) that can be connected to the hearing device 2, such as for example a fitness armband or a smartwatch. In the exemplary embodiment shown in FIG. 1, the hearing device 2 takes the form of a so-called ITE device, but the features and functionalities described on the basis of FIG. 1 are also readily transferable to a BTE device, an RIC device, a CIC device or other conceivable forms of configuration.

The hearing device 2 has a housing 4 with a top plate 6 which, when the hearing instrument 1 is worn as intended in an external auditory canal of a wearer (not shown), is facing the free space alongside the wearer's ear. In the region of the top plate 6, the hearing device 2 has at least one outer microphone M1, which is set up to convert an ambient sound signal 8 into an audio signal 10. In the region of the top plate 6, there may also be disposed a further microphone (not shown) for generating a further audio signal for directional processing of the two audio signals. The audio signal 10 is passed on to a signal processing device 12 of the hearing device 2, and is processed there according to the audiological requirements of the wearer of the hearing device 2, and may be amplified and/or compressed in this case in particular in terms of the frequency band. Similarly, a boosting of voice components in the audio signal 10 may take place in this case, and also a (possibly directional) noise suppression.

The processing of the audio signal 10 taking place in the way described in the signal processing device 12 has the effect of generating an output signal 14, which is converted by a loudspeaker 16 of the hearing device 2 into an output sound signal 18. The output sound signal 18, which preferably contains the correspondence of the sound signal 8 that is prepared user-specifically for the wearer of the hearing device 2, is output during operation of the device 2 as intended into the auditory canal of the wearer, which during wearing is at least partially and usually almost completely closed by the housing 4. The closing of the wearer's auditory canal (not shown) produces so-called inclusion effects, i.e. structure-borne sound that is output through the skin to the auditory canal and cannot escape because of the closure of the same, but is instead perceived to a considerable extent by the eardrum of the wearer of the hearing device 2.

Since dull noises, which may be perceived by the wearer as disturbing to unpleasant, can be produced in this case, for example when walking or when the jawbone is moving, the hearing device 2 has a module for actively suppressing occlusion effects (active occlusion cancellation, AOC) 20, which is implemented on a control unit 21 on which the signal processing device 12 is also implemented. A first input transducer 22 is also disposed in the region of the loudspeaker 16 for the AOC 20. The first input transducer 22 in the present case is configured as a microphone, and during operation of the device 2 as intended, is set up to record a first structure-borne sound signal 24, which is emitted in the auditory canal of the wearer, and to convert it into a first input signal 26. The occlusion effects can then be rectified on the basis of the first input signal 26, in that a corresponding compensation signal is mixed in with the output signal 14 by the AOC 20.

However, it is also possible on the basis of the first input transducer 22 to carry out a pulse measurement in a way still to be described. This includes using the first input signal 26, which contains the first structure-borne sound signal 24. Usually, sound components of a pulse beat of the veins that surround the auditory canal are also contained in the first structure-borne sound signal 24. On the basis of these sound components, a pulse rate can be measured, in that for example periodicities in the first input signal 26 are identified. Since the sound components of the pulse beat in the first structure-borne sound signal 24 are however overlaid by many other noises, and consequently sometimes there may be an unfavorable SNR, to assist the pulse measurement, the hearing device 2 also has a first sensor 30, which in the present case takes the form of a PPG sensor 32.

This PPG sensor 32 includes at least one light source 34 configured as an LED and also a light sensor 36 configured as a photodiode, which is disposed oppositely to the light source 34. The light source 34 and the light sensor 36 are connected in this case to the control unit 21, which on the one hand activates the output of outgoing light signals 35 into the tissue of the auditory canal by the light source 34, and on the other hand analyzes incoming light signals 37 registered by the light sensor 36 in the auditory canal. The outgoing light signals 35 into the skin, and consequently into the tissue of the auditory canal, thereby propagate partially through the tissue and also through the vessels in the auditory canal, and thereby undergo a modulation pulsating with the pulse beat. Subsequently, parts of the thus-modulated outgoing light signals 35 leave again laterally through the skin into the auditory canal, and are registered by the light sensor 36 as the incoming light signals 37. A pulse rate can then likewise be determined on the basis of the modulations of the registered light signals 37. However, because of the outgoing light signals 35, this determination by using the PPG sensor has a higher energy consumption than the aforementioned pulse measurement on the basis of the first input signal 26.

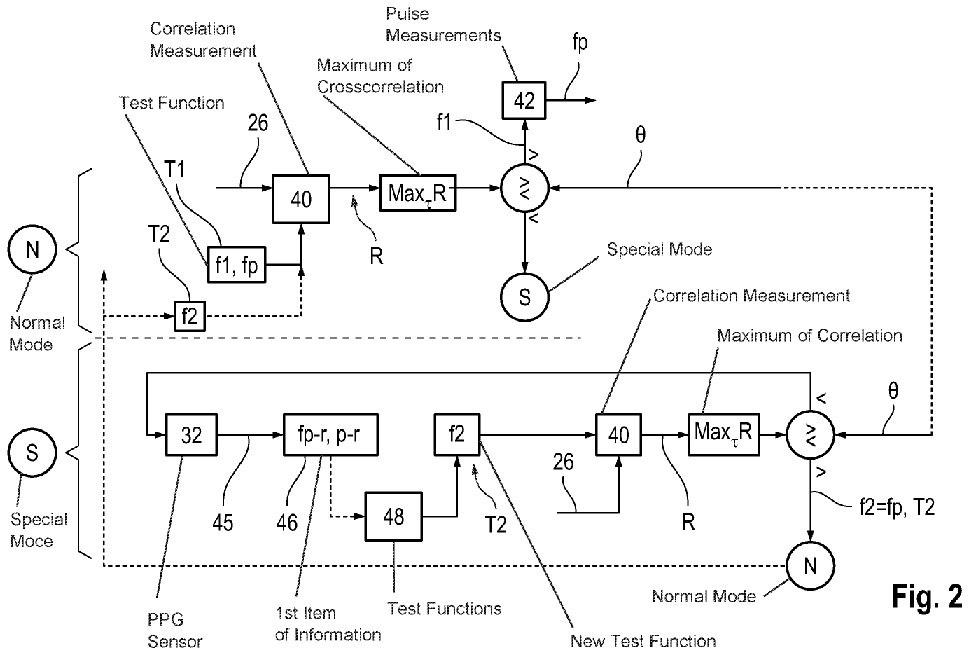

In FIG. 2, therefore, the sequence of an energy-efficient and at the same time reliable pulse measurement by using the hearing device as shown in FIG. 1 is shown in a block diagram. In a normal mode N, a correlation measurement 40 with respect to a test function T1 (a so-called correlator), to which a frequency f1 and a phase p1 are allocated (the frequency f1 may for example take the form of a periodicity of the test function T1, or be provided on the basis of an inverse duration of the test function T1 in the time domain), is thereby carried out for the first input signal 26, which as described is generated by the first input transducer 22 from the structure-borne sound signal 24, and contains sound components of a pulse beat. The selection of the test function T1 is further described below. For example, a crosscorrelation R of the first input signal 22 and the test function T1 is therefore calculated, and thereby forms a maximum Max_{τ} R with regard to a time argument τ of the crosscorrelation R. If the maximum Max_{τ} R of the crosscorrelation lies above a predetermined limit value θ, that is to say Max_{τ} R>θ, the correlation between the test function T1 and the first input signal 22 is found to be sufficient. In this case, the frequency f1 allocated to the test function T1 is output as a pulse rate fp of a pulse measurement 42. The phase p1 of the test function T1 is also assumed as the phase of the pulse beat measured in the pulse measurement 42.

If, however, the maximum Max_{τ} R of the crosscorrelation falls below the predetermined limit value θ, that is to say Max_{τ} R<θ, there is not a sufficient correlation between the test function T1 and the first input signal 22. The pulse measurement 42 by using the correlation with the test function T1 has then failed in the normal mode N, and a special mode S is initiated. In the special mode S, an auxiliary signal 45, which takes the form of the measurement signal of the incoming light signals 37, is measured by the PPG sensor 32 as the first sensor 30. From this auxiliary signal 45, a reference pulse rate fp-r can then be determined as a first item of information 46 about the pulse beat of the person. Moreover, a reference phase p-r may possibly also be determined. The determination of the reference pulse rate fp-r on the basis of the auxiliary signal 45 generated by the PPG sensor 32 has already been described further above.

On the basis of the reference pulse rate fp-r (and possibly on the basis of the reference phase p-r) it is then possible in the special mode S to select, from a plurality 48 of test functions, a new test function T2 as a new correlator, which is allocated the corresponding reference pulse rate fp-r (or which is allocated a frequency f2 that comes closest to the reference pulse rate fp-r).

Then, the correlation measurement 40 of the first input signal 26 is carried out with respect to the new test function T2, i.e., as described above, the associated crosscorrelation R is formed. If in this case the maximum Max_{τ} R lies above the predetermined limit value θ, the correlation between the new test function T2 and the first input signal 26 is regarded as sufficiently high. The frequency f2 assigned to the new test function T2 is output as the pulse rate fp, and then consequently provides the result of the pulse measurement 42. In the further course of the procedure, the pulse measurement can then take place again in the normal mode N, with the new test function T2 that has just been predetermined in the special mode S then being used as the correlator for the first input signal 26. In the case where the maximum of the crosscorrelation Max_{τ} R between the first input signal 26 and the new test function T2 lies below the predetermined limit value θ, in the special mode S the detection of the auxiliary signal 45 on the basis of the PPG sensor 32 and the subsequent determination of a reference pulse rate and also the associated selection of a further test function from the plurality 42 of given test functions are repeated.

In the way described, an initiation of the pulse measurement 42 may also take place in the special mode S, i.e. after starting operation or the like a selection of the suitable test function may first be carried out in the special mode S by using the PPG sensor 32, in order then, for the further course of the procedure, to carry out the pulse measurement 42 in the normal mode N just with the first input signal 26 (and the test function previously predetermined in the special mode S). Since the first input transducer 22 has a much lower power consumption in comparison with the PPG sensor 32, and correspondingly only consumes negligible battery power, the pulse measurement 42 in the normal mode N saves much more energy. In particular, it is also possible to change to the special mode S each time a change in the pulse rate (for example during sporting activity) has the effect that the correlation between the first input signal 26 and the corresponding test function “is lost” (that is to say falls below the predetermined limit value θ).

Although the invention has been illustrated and described more specifically in detail by the preferred exemplary embodiment, the invention is not restricted by the examples disclosed and other variations can be derived therefrom by a person skilled in the art without departing from the scope of protection of the invention.

The following is a summary list of reference numerals and the corresponding structure used in the above description of the invention.

### LIST OF REFERENCE SIGNS

1 Hearing instrument
2 Hearing device
4 Housing
6 Top plate
8 Sound signal
10 Audio signal
12 Signal processing device
14 Output signal
16 Loudspeaker
18 Output sound signal
20 AOC (active occlusion cancellation)
21 Control unit
22 First input transducer
24 First structure-borne sound signal
26 First input signal
30 First sensor
32 PPG sensor
34 Light source
35 Outgoing light signals
36 Light sensor
37 Incoming light signals
40 Correlation measurement
42 Pulse measurement
45 Auxiliary signal
46 First item of information
48 Plurality (of test functions)
f1, f2 Frequency (of the test function or of the new test function)
fp Pulse rate
fp-r Reference pulse rate
Max_{τ} R Maximum (of the crosscorrelation)
M1 Outer microphone
N Normal mode
p1 Phase (of the first test function)
p-r Reference phase
R Crosscorrelation
S Special mode
T1, T2 Test function
θ Predetermined limit value

1. A method for measuring the pulse of a person by using a hearing system, the method comprising:
providing at least one first hearing instrument with an electroacoustic first input transducer;
using the first input transducer to pick up a first structure-borne sound signal at an ear of the person and to generate a first input signal from the first structure-borne sound signal; and
determining a pulse rate based on an amplitude profile of the first input signal.

2. The method according to claim 1, which further comprises:
determining the pulse rate based on a first item of information; and
providing the first item of information with at least one of at least two extremes or at least two rising or falling flanks of an envelope of the first input signal or an absolute value function of the first input signal.

3. The method according to claim 1, which further comprises determining the pulse rate based on a correlation measurement of the first input signal.

4. The method according to claim 3, which further comprises determining a correlation of the first input signal with a test function, being assigned a predetermined frequency, for the correlation measurement.

5. The method according to claim 4, which further comprises:
providing a plurality of different test functions with different frequencies;
determining a correlation of the first input signal with a test function from the plurality of different test functions; and
determining a first test function from the plurality of different test functions based on a maximum of correlations.

6. The method according to claim 1, which further comprises:
in a normal mode, determining the pulse rate based on the amplitude profile of the first input signal;
in an event of an error when determining the pulse rate in the normal mode, changing to a special mode; and
in the special mode, obtaining a first item of information about a pulse beat of the person based on at least one of an auxiliary signal or a correlation measurement, and determining the pulse rate based on the first item of information.

7. The method according to claim 6, which further comprises, at least for a time or in the special mode:
using at least one of a first sensor of the hearing system or a second input transducer of the hearing system to generate an auxiliary signal;
obtaining the first item of information about the pulse beat of the person based on the auxiliary signal; and
then determining the pulse rate based on the first item of information and based on the first input signal.

8. The method according to claim 7, which further comprises:
determining a correlation of the first input signal with a test function, being assigned a predetermined frequency, for the correlation measurement; and
predetermining the frequency assigned to the test function based on the first item of information.

9. The method according to claim 8, which further comprises selecting the test function based on a plurality of different test functions with at least one of different frequencies or phases based on the first item of information.

10. The method according to claim 6, which further comprises:
providing a plurality of different test functions with different frequencies;
determining a correlation of the first input signal with a test function from the plurality of different test functions;
determining a first test function from the plurality of different test functions based on a maximum of correlations; and
in the special mode, at least one of:
determining the first test function from the plurality of test functions, or
determining a frequency of the first test function based on a maximum of correlations as the first item of information.

11. The method according to claim 6, which further comprises, after a successful determination of the pulse rate in the special mode, changing back to the normal mode.

12. The method according to claim 11, which further comprises, after the change from a previous special mode to the normal mode, determining the pulse rate based on the first input signal and the first item of information obtained in the previous special mode.

13. The method according to claim 12, which further comprises:
determining the pulse rate based on a correlation measurement of the first input signal;
in the normal mode, respectively carrying out a correlation measurement with the first input signal for a group of test functions having frequencies forming a corridor around the frequency of the first test function; and
updating the frequency of the first test function based on a maximum of correlation measurements.

14. The method according to claim 3, which further comprises obtaining the first item of information when the value of the correlation measurement falls below a predetermined limit value.

15. The method according to claim 1, which further comprises using the first input transducer to pick up the first structure-borne sound signal in an auditory canal at the ear of the person.

16. The method according to claim 1, which further comprises using an electroacoustic second input transducer of a second hearing instrument of the hearing system to pick up a second structure-borne sound signal at the other ear of the person, and generating the auxiliary signal as a second input signal from the second structure-borne sound signal.

17. The method according to claim 16, which further comprises carrying out a correlation measurement of the first and second input signals, and determining the pulse rate based on a value of a correlation resulting from the correlation measurement of the first and second input signals.

18. The method according to claim 7, which further comprises:
using a photoplethysmography sensor as the first sensor;
carrying out a pulse measurement based on the auxiliary signal; and
determining a reference pulse rate of the person as a first item of information, and then using the first item of information as a reference for a subsequent determination of the pulse rate based on the first input signal or only based on the first input signal.

19. The method according to claim 1, which further comprises:
at least one of using an acceleration sensor disposed on a carotid artery as the first sensor or using an electrocardiogram sensor as the first sensor and creating an electrocardiogram of the person as an auxiliary signal;
determining a reference pulse rate of the person as a first item of information based on a movement of the carotid artery recorded by the acceleration sensor or from the electrocardiogram; and
then using the first item of information as a reference for a subsequent determination of the pulse rate based on the first input signal or only based on the first input signal.

20. A hearing system, comprising:
at least a first hearing instrument with an electroacoustic first input transducer; and
a control unit configured to carry out the method for measuring the pulse of a person according to claim 1, when the first hearing instrument is worn at an ear of the person.
